SEQUENCE LISTING (59 sequences)

SEQ ID NO: 1-2 - skipped (2 entries)

SEQ ID NO: 3 - protein, 18 aa, synthetic construct
  REGION 1..18: note = Rigid linker 1
EAAAAKEAAAAKEAAAAK

SEQ ID NO: 4 - protein, 613 aa, Pseudomonas aeruginosa
  DOMAIN 1..252: note = Receptor-binding domain
  DOMAIN 253..364: note = Translocation domain
  DOMAIN 405..613: note = ADP-ribosyltransferase domain
  REGION 1..613: note = Full length Pseudomonas Exotoxin A (PE)
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 5 - protein, 26 aa, synthetic construct
GWEQLEQCGYPVQRLVALYLAARLSW

SEQ ID NO: 6 - protein, 34 aa, synthetic construct
  REGION 1..34: note = PE translocation peptide (PE280-313)
GWEQLEQCGYPVQRLVALYLAARLSWNQVDQVIR

SEQ ID NO: 7 - protein, 46 aa, synthetic construct
  REGION 1..46: note = PE translocation peptide (PE268-313)
PLETFTRHRQPRGWEQLEQCGYPVQRLVALYLAARLSWNQVDQVIR

SEQ ID NO: 8 - protein, 61 aa, synthetic construct
  REGION 1..61: note = PE translocation peptide (PE253-313)
GGSLAALTAHQACHLPLETFTRHRQPRGWEQLEQCGYPVQRLVALYLAARLSWNQVDQVIR

SEQ ID NO: 9 - protein, 112 aa, synthetic construct
  REGION 1..112: note = PE translocation peptide (PE253-364)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAN

SEQ ID NO: 10 - protein, 293 aa, Shigella dysenteriae
  REGION 1..293: note = Full length Shiga toxin (Stx) subunit A
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 11 - protein, 293 aa, Escherichia coli
  REGION 1..293: note = Full length Shiga-like toxin I (Slt-I) subunit A
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 12 - protein, 8 aa, synthetic construct
  REGION 1..8: note = Stx translocation peptide (Stx240-247, minimal)
LNCHHHAS

SEQ ID NO: 13 - protein, 12 aa, synthetic construct
  REGION 1..12: note = Stx translocation peptide (Stx240-251)
LNCHHHASRVAR

SEQ ID NO: 14 - protein, 37 aa, synthetic construct
  REGION 1..37: note = Stx translocation peptide (Stx211-247)
PDYHGQDSVRVGRISFGSINAILGSVALILNCHHHAS

SEQ ID NO: 15 - protein, 41 aa, synthetic construct
  REGION 1..41: note = Stx translocation peptide (Stx211-251)
PDYHGQDSVRVGRISFGSINAILGSVALILNCHHHASRVAR

SEQ ID NO: 16 - protein, 84 aa, synthetic construct
  REGION 1..84: note = Stx translocation peptide (Stx168-251)
ALRFRQIQRGFRTTLDDLSGRSYVMTAEDVDLTLNWGRLSSVLPDYHGQDSVRVGRISFGSINAILGSVALILNCHHHASRVAR

SEQ ID NO: 17 - protein, 261 aa, Homo sapiens
  REGION 1..261: note = Full length CD40 ligand (CD40L1-261)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - protein, 215 aa, synthetic construct
  REGION 1..215: note = Truncated CD40 ligand (CD40L47-261)
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 19 - protein, 154 aa, synthetic construct
  REGION 1..154: note = Truncated CD40 ligand (CD40L108-261, also referred to as 18sCD40L)
ENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 246 aa, synthetic construct
  REGION 1..246: note = Anti-CD40 scFv (VH-L-VL)
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 21 - protein, 246 aa, synthetic construct
  REGION 1..246: note = Anti-CD40 scFv (VL-L-VH)
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGGGGGSGGGGSGGGGSEVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 22 - protein, 119 aa, synthetic construct
  REGION 1..119: note = VH of the anti-CD40 scFv
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 23 - protein, 112 aa, synthetic construct
  REGION 1..112: note = VL of the anti-CD40 scFv
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 24 - protein, 10 aa, synthetic construct
  REGION 1..10: note = VH CDR1
GFTFSTYGMH

SEQ ID NO: 25 - protein, 26 aa, synthetic construct
  REGION 1..26: note = VH CDR2
GKGLEWLSYISGGSSYIFYADSVRGR

SEQ ID NO: 26 - protein, 13 aa, synthetic construct
  REGION 1..13: note = VH CDR3
CARILRGGSGMDL

SEQ ID NO: 27 - protein, 15 aa, synthetic construct
  REGION 1..15: note = VL CDR1
CTGSSSNIGAGYNVY

SEQ ID NO: 28 - protein, 7 aa, synthetic construct
  REGION 1..7: note = VL CDR2
GNINRPS

SEQ ID NO: 29 - protein, 12 aa, synthetic construct
  REGION 1..12: note = VL CDR3
CAAWDKSISGLV

SEQ ID NO: 30 - protein, 4 aa, synthetic construct
  REGION 1..4: note = ER retention sequence
KDEL

SEQ ID NO: 31 - protein, 12 aa, synthetic construct
  REGION 1..12: note = ER retention sequence
KKDLRDELKDEL

SEQ ID NO: 32 - protein, 13 aa, synthetic construct
  REGION 1..13: note = ER retention sequence
KKDELRDELKDEL

SEQ ID NO: 33 - protein, 13 aa, synthetic construct
  REGION 1..13: note = ER retention sequence
KKDELRVELKDEL

SEQ ID NO: 34 - protein, 12 aa, synthetic construct
  REGION 1..12: note = ER retention sequence
KDELKDELKDEL

SEQ ID NO: 35 - protein, 28 aa, synthetic construct
  REGION 1..28: note = CD28 consensus sequence
  SITE 8: note = X is I or L
  SITE 10: note = X is V, F or A
  SITE 11: note = X is M or L
  SITE 17: note = X is L or I
TDIYFCKXEXXYPPPYXDNEKSNGTIIH

SEQ ID NO: 36 - protein, 28 aa, synthetic construct
  REGION 1..28: note = CD28-activating peptide (minimal)
TDIYFCKIEVMYPPPYLDNEKSNGTIIH

SEQ ID NO: 37 - protein, 53 aa, synthetic construct
  REGION 1..53: note = CD28-activating peptide
NCDGKLGNESVTFYLQNLYVNQTDIYFCKIEVMYPPPYLDNEKSNGTIIHVKG

SEQ ID NO: 38 - protein, 6 aa, synthetic construct
  REGION 1..6: note = Rigid linker
EAAAAK

SEQ ID NO: 39 - protein, 194 aa, African swine fever virus
  REGION 1..194: note = Antigen ASFV CP204L
MDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKK

SEQ ID NO: 40 - protein, 184 aa, African swine fever virus
  REGION 1..184: note = Antigen ASFV E183L
MDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSL

SEQ ID NO: 41 - protein, 408 aa, synthetic construct
  REGION 1..408: note = Antigen ASFV CP204L-E183L
MDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKASAEAAAAKEAAAAKEAAAAKEAAAAKATSMDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSL

SEQ ID NO: 42 - protein, 331 aa, Classical Swine Fever Virus
  REGION 1..331: note = Antigen CSFV E2
RLACKEDYRYAISSTNEIGLLGAGGLTTTWKEYSHDLQLNDGTVKAICVAGSFKVTALNVVSRRYLASLHKGALLTSVTFELLFDGTNPSTEEMGDDFGFGLCPFDTSPVVKGKYNTTLLNGSAFYLVCPIGWTGVIECTAVSPTTLRTEVVKTFRREKPFPHRMDCVTTTVENEDLFYCKLGGNWTCVKGEPVVYTGGQVKQCKWCGFDFNEPDGLPHYPIGKCILANETGYRIVDSTDCNRDGVVISAEGSHECLVGNTTVKVHASDERLGPMPCRPKEIVSSAGPVRKTSCTFNYAKTLKNKYYEPRDSYFQQYMLKGEYQYWFDLDV

SEQ ID NO: 43 - protein, 192 aa, Classical Swine Fever Virus
  REGION 1..192: note = Antigen CSFV NS3p
GPAVCKKVTEHEKCTTSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKL

SEQ ID NO: 44 - protein, 553 aa, synthetic construct
  REGION 1..553: note = Antigen CSFV E2-NS3p
RLACKEDYRYAISSTNEIGLLGAGGLTTTWKEYSHDLQLNDGTVKAICVAGSFKVTALNVVSRRYLASLHKGALLTSVTFELLFDGTNPSTEEMGDDFGFGLCPFDTSPVVKGKYNTTLLNGSAFYLVCPIGWTGVIECTAVSPTTLRTEVVKTFRREKPFPHRMDCVTTTVENEDLFYCKLGGNWTCVKGEPVVYTGGQVKQCKWCGFDFNEPDGLPHYPIGKCILANETGYRIVDSTDCNRDGVVISAEGSHECLVGNTTVKVHASDERLGPMPCRPKEIVSSAGPVRKTSCTFNYAKTLKNKYYEPRDSYFQQYMLKGEYQYWFDLDVASAEAAAAKEAAAAKEAAAAKEAAAAKATSGPAVCKKVTEHEKCTTSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKL

SEQ ID NO: 45 - protein, 192 aa, Porcine Circovirus 2
  REGION 1..192: note = Antigen PCV2 ORF2
NGIFNTRLSRTFGYTIKRTTVKTPSWAVDMMRFNINDFLPPGGGSNPRSVPFEYYSISKVKVEFWPCSPITQGDSGVGSSAVILDDNFVTKATALTYDPYVNYSSRHTITQPFSYHSRYFTPKPVLDSGGGAAAPNNKRNQLWLRLQTAGNVDHVGLGTAFENSIYDQEYNIRVTMYVQFREFNLKDPPLNP

SEQ ID NO: 46 - protein, 708 aa, Porcine Epidemic Diarrhea Virus
  REGION 1..708: note = Antigen PEDV S1
CSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFFKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPG

SEQ ID NO: 47 - protein, 60 aa, Porcine Reproductive and Respiratory Syndrome Virus
  REGION 1..60: note = Antigen PRRSV D (comprising one repeat of the C-terminal portion of PRRSV ORF7)
RHHFTPSERQLCLSSIQTAFNQGAGTCILSDSGRISYTVEFSLPTHHTVRLIRVTAPPSA

SEQ ID NO: 48 - protein, 220 aa, synthetic construct
  REGION 1..220: note = Antigen PRRSV DGD (comprising two repeats of the C-terminal portion of PRRSV ORF7)
RHHFTPSERQLCLSSIQTAFNQGAGTCILSDSGRISYTVEFSLPTHHTVRLIRVTAPPSALDQVIRNALASPGSGGDLGEAIREQPEQARLALTLAAAESERFVRQGTGNDEAGAANADVVSLTCPVAAGECAGPADSGDALLERNYPTGAEFLGDGGDVRHHFTPSERQLCLSSIQTAFNQGAGTCILSDSGRISYTVEFSLPTHHTVRLIRVTAPPSA

SEQ ID NO: 49 - protein, 165 aa, synthetic construct
  REGION 1..165: note = Antigen PRRSV M (M12) (from fusion of the C-terminal portion of NSP 10 and the N-terminal portion of NSP 11)
NNKECTVAQALGNGDKFRATDKRVVDSLRAICADLEGSSSPLPKVAHNLGFYFSPDLTQFAKLPIELAPHWPVVSTQNNEKWPDRLVASLRPLDKYSRACIGAGYMVGPSVFLGTPGVVSYYLTKFVKGEAQVLPETVFSTGRIEVDCREYLDDREREVAASLPH

SEQ ID NO: 50 - protein, 62 aa, synthetic construct
  REGION 1..62: note = Antigen PRRSV R (RSAB) (from fusion of the N-terminal portion of PRRSV ORF6 and the N-terminal portion of ORF5)
MGSLDDFCNDSTAAQKLVLAFSITYTPIFVAGGSSSTYQYIYNLTICELNGTDWLSNHFDWA

SEQ ID NO: 51 - protein, 58 aa, synthetic construct
  REGION 1..58: note = Antigen PRRSV P (PQAB) (another fusion antigen from fusion of the N-terminal portion of PRRSV ORF6 and the N-terminal portion of ORF5)
GSSLDDFCYDSTAPQKVLLAFSITYASNDSSSHLQLIYNLTLCELNGTDWLANKFDWA

SEQ ID NO: 52 - protein, 652 aa, synthetic construct
  REGION 1..652: note = Fusion protein CD40L-TPE-CP204L-E183L
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMTSMDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKASAEAAAAKEAAAAKEAAAAKEAAAAKATSMDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSLASTRAKDEL

SEQ ID NO: 53 - protein, 438 aa, synthetic construct
  REGION 1..438: note = Fusion protein CD40L-TPE-CP204L
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMTSMDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKASTRAKDEL

SEQ ID NO: 54 - protein, 428 aa, synthetic construct
  REGION 1..428: note = Fusion protein CD40L-TPE-E183L
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMTSMDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSLASTRAKDEL

SEQ ID NO: 55 - protein, 797 aa, synthetic construct
  REGION 1..797: note = Fusion protein CD40L-TPE-E2-NS3p
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMTSRLACKEDYRYAISSTNEIGLLGAGGLTTTWKEYSHDLQLNDGTVKAICVAGSFKVTALNVVSRRYLASLHKGALLTSVTFELLFDGTNPSTEEMGDDFGFGLCPFDTSPVVKGKYNTTLLNGSAFYLVCPIGWTGVIECTAVSPTTLRTEVVKTFRREKPFPHRMDCVTTTVENEDLFYCKLGGNWTCVKGEPVVYTGGQVKQCKWCGFDFNEPDGLPHYPIGKCILANETGYRIVDSTDCNRDGVVISAEGSHECLVGNTTVKVHASDERLGPMPCRPKEIVSSAGPVRKTSCTFNYAKTLKNKYYEPRDSYFQQYMLKGEYQYWFDLDVASAEAAAAKEAAAAKEAAAAKEAAAAKATSGPAVCKKVTEHEKCTTSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLASTRAKDEL

SEQ ID NO: 56 - protein, 659 aa, synthetic construct
  REGION 1..659: note = Fusion protein CP204L-E183L-TStx-CD40L
MKLTMGSSHHHHHHSSGLVPRGSHMTSMDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKASAEAAAAKEAAAAKEAAAAKEAAAAKATSMDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSLASTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 57 - protein, 445 aa, synthetic construct
  REGION 1..445: note = Fusion protein CP204L-TStx-CD40L
MKLTMGSSHHHHHHSSGLVPRGSHMTSMDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKKASTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 58 - protein, 433 aa, synthetic construct
  REGION 1..433: note = Fusion protein E183L-TStx-CD40L
MKLTMGSSHHHHHHSSGLVPRGSHMMDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSLASTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 59 - protein, 804 aa, synthetic construct
  REGION 1..804: note = Fusion protein E2-NS3p-TStx-CD40L
MKLTMGSSHHHHHHSSGLVPRGSHMTSRLACKEDYRYAISSTNEIGLLGAGGLTTTWKEYSHDLQLNDGTVKAICVAGSFKVTALNVVSRRYLASLHKGALLTSVTFELLFDGTNPSTEEMGDDFGFGLCPFDTSPVVKGKYNTTLLNGSAFYLVCPIGWTGVIECTAVSPTTLRTEVVKTFRREKPFPHRMDCVTTTVENEDLFYCKLGGNWTCVKGEPVVYTGGQVKQCKWCGFDFNEPDGLPHYPIGKCILANETGYRIVDSTDCNRDGVVISAEGSHECLVGNTTVKVHASDERLGPMPCRPKEIVSSAGPVRKTSCTFNYAKTLKNKYYEPRDSYFQQYMLKGEYQYWFDLDVASAEAAAAKEAAAAKEAAAAKEAAAAKATSGPAVCKKVTEHEKCTTSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLASTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2024/0141016 A1
(43) Pub. Date: May 2, 2024

(54) IMMUNOGENIC FUSION PROTEINS AGAINST INFECTIOUS ANIMAL DISEASES

(71) Applicant: NAVICURE BIOPHARMACEUTICALS LIMITED, Apia City (WS)

(72) Inventor: Chia-Mao WU, Hsinchu City (TW)

(21) Appl. No.: 18/270,856

(22) PCT Filed: Oct. 28, 2022
(86) PCT No.: PCT/US2022/078832

§ 371 Date: Jul. 4, 2023

(51) Int. Cl. C07K 14/705; A61P 37/02; C12N 15/62

(52) U.S. Cl. CPC C07K 14/70578; A61P 37/02; C07K 14/70521; C12N 15/62; A61K 38/00; C07K 2319/01; C07K 2319/50; C07K 2319/55

(57) ABSTRACT

Immunogenic fusion proteins against infectious animal diseases. A fusion protein is disclosed, which comprises a CD40-binding domain; an antigen of a pathogen; a translocation domain located between the CD40-binding domain and the antigen, and a furin and/or cathepsin L cleavage site located between the CD40-binding domain and the translocation domain. Also disclosed are pharmaceutical compositions, expression vectors and use of the fusion proteins of the invention for eliciting an antigen-specific cell-mediated immune response, or for reducing, inhibiting, treating and/or ameliorating an infectious animal disease caused by a pathogen in an animal in need thereof.

### REFERENCE TO AN ELECTRONIC SEQUENCE LISTING

The contents of the electronic sequence listing (10040-004PCT_sequence listing_ST26.xml; size 84 KB; and creation date Oct. 24, 2022) is herein incorporated by reference in its entirety.

### FIELD OF THE INVENTION

The present invention relates to fusion proteins, and more specifically to immunogenic fusion proteins for eliciting antigen-specific cell-mediated immune responses against infectious animal diseases.

### BACKGROUND OF THE INVENTION

The adaptive immune system includes both humoral and cell-mediated immunities, both of which destroy invading pathogens. B- and T-lymphocytes are responsible for antibody and cell-mediated immune responses, respectively. Adaptive immunity against pathogens leads to enhanced immune responses to future encounters. Several adaptive immunity therapy strategies have been evaluated in the clinical settings. There is, however, still a need for developing new therapeutic vaccines for treating infectious animal diseases caused by pathogens.

### SUMMARY OF THE INVENTION

In one aspect, the invention relates to a fusion protein comprising: (a) a CD40-binding domain; (b) an antigen of a pathogen; (c) a translocation domain, located between the CD40-binding domain and the antigen; and (d) a furin and/or cathepsin L cleavage site, located between the CD40-binding domain and the translocation domain, wherein the pathogen is at least one selected from the group consisting of Classical Swine Fever Virus (CSFV), African Swine Fever Virus (ASFV), Porcine Circovirus 2 (PCV2), Porcine Reproductive and Respiratory Syndrome Virus (PRRSV), Porcine Epidemic Diarrhea Virus (PEDV), Foot-and-Mouth Disease Virus (FMDV), Swine Vesicular Disease Virus (SVDV), Pseudorabies Virus (PRV), Transmissible Gastroenteritis Virus (TGEV), Mycoplasma hyopneumoniae, Newcastle Disease Virus (NDV), Infectious Bronchitis Virus (IBV), Infectious Bursal Disease Virus (IBDV), Parvovirus, Poxvirus, Rotavirus, and Influenza Virus.

In another aspect, the invention relates to a DNA fragment, or an expressing vector comprising a DNA fragment encoding a fusion protein of the invention.

The invention further relates to a pharmaceutical or a vaccine composition comprising a fusion protein of the invention and a pharmaceutical acceptable carrier and/or an adjuvant.

Yet in another aspect, the invention relates to use of a fusion protein, a pharmaceutical composition, or a vaccine composition of the invention in the manufacture of a medicament for eliciting an antigen-specific cell-mediated immune response, or for reducing, inhibiting, treating, and/or ameliorating an infectious animal disease caused by a pathogen in an animal in need thereof.

The invention also relates to a fusion protein, a pharmaceutical composition, or a vaccine composition of the invention for use in eliciting an antigen-specific cell-mediated immune response, or for use in reducing, inhibiting, treating, and/or ameliorating an infectious animal disease caused by a pathogen in an animal in need thereof.

Alternatively, the invention relates to a method for eliciting an antigen-specific cell-mediated immune response, or for reducing, inhibiting, treating, and/or ameliorating an infectious animal disease caused by a pathogen in an animal in need thereof, said method comprising administering an effective amount of a fusion protein, a pharmaceutical composition or a vaccine composition of the invention to the animal in need thereof.

### BRIEF DESCRIPTION OF DRAWINGS

FIGS. 1A-E and 2A-E are vector maps of the invention.

FIGS. 3A-H are schematic drawings illustrating immunogenic fusion proteins in various embodiments of the invention.

FIGS. 4-6 are graphs illustrating the inductions of IFN-γ, IL-2, and TNF-α, respectively, in CD4⁺ memory T cells from each animal group.

FIGS. 7A-B are graphs showing IFN-γ⁺ immunospots in the splenocytes from each animal group.

FIGS. 8A-B are graphs illustrating the serum antigen-specific antibody levels on Day 21 in each animal group. The antibody levels were assayed by ELISA using CD40L-T^{PE}-E2-NS3p fusion protein as a coating protein.

FIGS. 9A-B are graphs illustrating the serum antigen-specific antibody levels on Day 21 in each animal group. The antibody levels were assayed by ELISA using E2-NS3p peptide as a coating protein.

FIGS. 10A-C are graphs illustrating the inductions of IFN-γ, IL-2, and TNF-α, respectively, in CD4⁺ memory T cells from each animal group with an extended dosing schedule.

FIG. 11 is a graph illustrating IFN-γ⁺ immunospots in the splenocytes from each animal group with an extended dosing schedule.

FIG. 12 is a graph illustrating the serum antigen-specific antibody levels in each animal group with an extended dosing schedule.

### DETAILED DESCRIPTION OF THE INVENTION

### Definitions

Professional APCs and non-professional APCs use an MHC class I molecule to display endogenous peptides on the cell membrane. These peptides originate within the cell itself, in contrast to the exogenous antigen displayed by professional APCs using MHC class II molecules. Cytotoxic CD8⁺ T cells can interact with antigens presented by the MHC class I molecule.

CD40 is a costimulatory protein expressed on antigen-presenting cells (e.g., dendritic cells, macrophages, and B cells). The binding of CD40L to CD40 activates antigen-presenting cells and induces a variety of downstream effects. CD40 is a drug target for cancer immunotherapy.

The term “a CD40-binding domain” refers to a protein that can recognize and binds to CD40. A CD40-binding domain may be selected from one of the following: “CD40 ligand (CD40L) or a functional fragment thereof”, “an anti-CD40 antibody or a functional fragment thereof.”

The terms “CD40L”, “CD40 ligand” and “CD154” are interchangeable. CD40L binds to CD40 (protein) on antigen-presenting cells (APC), which leads to many effects depending on the target cell type. CD40L plays a significant role in co-stimulation and regulation of the immune response via T cell priming and activation of CD40-expressing immune cells. U.S. Pat. No. 5,962,406 discloses the nucleotide and amino acid sequence of CD40L.

The terms “anti-CD40 antibody”, “CD40-specific antibody”, and “an antibody specifically against CD40” are interchangeable.

When the term “consist substantially of” is used in describing an amino acid sequence of a polypeptide, it means that the polypeptide may or may not have a starting amino acid “M” (translated from a start codon AUG) at N-terminal as a part of the polypeptide, depending on protein translation requirements. For example, when the second antigen fused to the first antigen, the starting amino acid “M” of the second antigen could be omitted or kept.

As used herein, “a translocation domain” is a polypeptide having biological activity in translocating a fused or linked antigen across an endosomal membrane into cytosol of a cell. The translocation domain guides or facilitates the antigen toward class I major histocompatibility complex (MHC-1) pathway (i.e., a cytotoxic T cell pathway) for antigen presentation.

The term “a Pseudomonas Exotoxin A (PE) translocation peptide (T^{PE})” refers to a PE domain II peptide or a functional fragment thereof that has the biological activity in translocation.

The term “a Shiga toxin (Stx) translocation peptide (T^{Stx})” refers to a Stx translocating domain or a functional fragment thereof that has the biological activity in translocation.

The terms “furin and/or cathepsin L” or “furin/cathepsin L” are interchangeable.

The term “furin and/or cathepsin L cleavage site” refers to a short peptide sequence having at least four amino acids that can be cleaved by furin or cathepsin L, or by both furin and cathepsin L. Said cleavage site is a furin and/or cathepsin L protease sensitive site. It may be a peptide linker comprising said cleavage site that is introduced into the fusion protein. In addition, the furin and/or cathepsin L cleavage site may be a PE or Stx intrinsic protease cleavage site present in or adjacent to the translocation domain of the fusion protein.

The terms “antigen” and “immunogen” are interchangeable. An antigen refers to an antigenic protein or polypeptide derived from a pathogen. Said antigen comprises at least one epitope for inducing desirable immune response. In some embodiments, the antigen is a polypeptide of at least 8 amino acids in length derived from a pathogen selected from the group consisting of Porcine Reproductive and Respiratory Syndrome Virus (PRRSV), African Swine Fever Virus (ASFV), Classical Swine Fever Virus (CSFV), Porcine Circovirus 2 (PCV2), Foot-and-Mouth Disease Virus (FMDV), Porcine Epidemic Diarrhea Virus (PEDV), Swine Vesicular Disease Virus (SVDV), Pseudorabies Virus (PRV), Transmissible Gastroenteritis Virus (TGEV), Mycoplasma hyopneumoniae, Newcastle Disease Virus (NDV), Infectious Bronchitis Virus (IBV), Infectious Bursal Disease Virus (IBDV), Parvovirus, Poxvirus, Rotavirus, and Influenza Virus.

CD28 (Cluster of Differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD28 is the receptor for CD80 (B7.1) and CD86 (B7.2) proteins. When activated by Toll-like receptor ligands, the CD80 expression is upregulated in antigen-presenting cells (APCs). CD28 is the only B7 receptor constitutively expressed on naive T cells. Association of the TCR of a naive T cell with MHC:antigen complex without CD28:B7 interaction results in a T cell that is anergic.

The term “an effective amount” refers to the amount of an active fusion protein that is required to confer a therapeutic effect on the treated subject. Effective doses will vary, as recognized by those skilled in the art, depending on rout of administration, excipient usage, and the possibility of co-usage with other therapeutic treatment.

The term “treating”, or “treatment” refers to administration of an effective amount of the fusion protein to a subject in need thereof, who has cancer or infection, or a symptom or predisposition toward such a disease, with the purpose of cure, alleviate, relieve, remedy, ameliorate, or reduce, inhibit the disease, the symptoms of it, or the predisposition towards it. Such a subject can be identified by a health care professional based on results from any suitable diagnostic method.

By “0 to 12 repeats” or “2 to 6 repeats”, it means that all integer unit amounts within the range “0 to 12” or “2 to 6” are specifically disclosed as part of the invention. Thus, 0, 1, 2, 3, 4, . . . 10, 11 and 12” or “2, 3, 4, 5 and 6” unit amounts are included as embodiments of this invention.

Abbreviations: MCS, multiple cloning sites; Rap1, Ras-proximate-1 or Ras-related protein 1; CD40, Cluster of differentiation 40; CDR, Complementarity-determining region; s.c., subcutaneously; a.a., amino acid.

### Fusion Proteins

The invention relates to a fusion protein comprising: (a) a CD40-binding domain; (b) an antigen of a pathogen; (c) a translocation domain, located between the CD40-binding domain and the antigen; and (d) a furin and/or cathepsin L cleavage site, located between the CD40-binding domain and the translocation domain, wherein the pathogen is selected from the group consisting of Classical Swine Fever Virus (CSFV), African Swine Fever Virus (ASFV), Porcine Circovirus 2 (PCV2), Porcine Reproductive and Respiratory Syndrome Virus (PRRSV), Porcine Epidemic Diarrhea Virus (PEDV), Foot-and-Mouth Disease Virus (FMDV), Swine Vesicular Disease Virus (SVDV), Pseudorabies Virus (PRV), Transmissible Gastroenteritis Virus (TGEV), Mycoplasma hyopneumoniae, Newcastle Disease Virus (NDV), Infectious Bronchitis Virus (IBV), Infectious Bursal Disease Virus (IBDV), Parvovirus, Poxvirus, Rotavirus, and Influenza Virus.

The fusion proteins of the invention can elicit an antigen-specific T cell immune response via MHC class I antigen presentation pathway. They share a common mechanism of action. Using CD40L-T^{PE}-Ag (Ag stands for any suitable antigen) as an example, the mechanism of action is as follows:
(1) CD40L-T^{PE}-AS binds to a CD40-expressing cell (e.g., dendritic cell or macrophage) and is internalized via a CD40-mediated endocytosis;
(2) CD40L-T^{PE}-Ag is cleaved by furin protease and/or cathepsin L protease within an endosome to remove the CD40L fragment away from the T^{PE}-Ag fragment;
(3) the T^{PE}-Ag fragment is translocated across the endosomal membrane and enter the cytosol;
(4) the T^{PE}-Ag fragment is digested by cytosol proteasomes to generate small antigens comprising epitopes;
(5) the small antigens are delivered via MHC class I pathway for antigen presentation; and
(6) a CD8⁺ T cell specific immune response is induced or enhanced by T-cell recognizing these presented antigens.

The same mechanism of action applies to Ag-T^{Stx}-CD40L, in which furin and/or cathepsin L protease cleavage would remove Ag-T^{Stx} fragment away from the CD40L fragment. Thus, the Ag-T^{Stx} fragment would be translocated across the endosomal membrane, enter the cytosol, digested by cytosol proteasomes to generate small antigens comprising epitopes. The small antigens would be delivered via MHC class I pathway for antigen presentation and a CD8⁺ T cell specific immune response would be induced or enhanced by T-cell recognizing these presented antigens.

No furin and/or cathepsin L cleavage site is present between the antigen and the translocation domain in the fusion protein of the invention. The presence of a furin and/or cathepsin L cleavage site and its location in the fusion protein permits removal of CD40-binding domain from the fusion protein after the furin and/or cathepsin L cleavage.

In one embodiment, the furin and/or cathepsin L cleavage site comprises or consists of 4-20 amino acids, preferred 4-10 amino acids, and more preferred 4-6 amino acids. In another embodiment, a furin and/or cathepsin L cleavage site comprises, consist of, or is SEQ ID NO: 1 or 2.

In another embodiment, the fusion protein of the invention further comprises a peptide linker between the CD40-binding domain and the translocation domain, wherein the furin and/or cathepsin L cleavage site is present in said peptide linker. The peptide linker may comprise (a) a rigid linker (EAAAAK)ₙ or (SEQ ID NO: 38)ₙ; and (b) a cleavable linker comprising a furin and/or cathepsin L cleavage site, wherein n is an integer from 0-12, preferably from 2-6, more preferably from 3-4, and said furin and/or cathepsin L cleavage site comprises SEQ ID NO: 1 or 2. In one embodiment, the peptide linker comprises (EAAAAK)₃ and RX¹RX²X³R (SEQ ID NO: 2; wherein X¹ is A, X² is Y, X³ is K). In another embodiment, the peptide linker comprises RX¹X²R (SEQ ID NO: 1; wherein X¹ is V, X² is A) and (EAAAAK)₃.

The translocation domain may be selected from a Pseudomonas Exotoxin A (PE) or a Shiga toxin (Stx). In one embodiment, the translocation domain comprises or is a Pseudomonas Exotoxin A (PE) translocation peptide (T^{PE}), with the proviso that the CD40-binding domain is located at the N-terminal of the fusion protein. In another embodiment, the translocation domain comprises or is a Shiga toxin (Stx) translocation peptide (T^{Stx}), with the proviso that the antigen is located at the N-terminal of the fusion protein.

In one embodiment, a fusion protein of the invention sequentially (from N- to C-terminal) comprises: (a) a CD40-binding domain; (b) a furin and/or cathepsin L cleavage site; (c) a translocation domain comprising a PE translocation peptide (T^{PE}); and (d) an antigen of a pathogen.

In another embodiment, the fusion protein of the invention sequentially (from N- to C-terminal) comprises: (a) a CD40-binding domain; (b) a peptide linker comprising a furin and/or cathepsin L cleavage site; (c) a translocation domain comprising a PE translocation peptide (T); and (d) an antigen of a pathogen.

In another embodiment, the fusion protein of the invention sequentially (from N- to C-terminal) comprises: (a) an antigen of a pathogen; (b) a translocation domain comprising a Stx translocation peptide (T^{Stx}); (c) a furin and/or cathepsin L cleavage site; and (d) a CD40-binding domain.

In another embodiment, the fusion protein of the invention sequentially (from N- to C-terminal) comprises: (a) an antigen of a pathogen; (b) a translocation domain comprising a Stx translocation peptide (T^{Stx}); (c) a peptide linker comprising a furin and/or cathepsin L cleavage site; and (d) a CD40-binding domain.

The T^{PE} or T^{Stx} is a functional moiety having a biological activity in translocation. The furin and/or cathepsin L cleavage site may be one selected from SEQ ID NO: 1, SEQ ID NO: 2 or an intrinsic furin cleavage site within, or derived from, PE or Stx.

In one embodiment, the PE translocation peptide (T^{PE}) is domain II (a.a. residues 253-364; SEQ ID NO: 9) of Pseudomonas Exotoxin A protein (full-length PE, SEQ ID NO: 4) or a functional moiety thereof.

In another embodiment, the PE translocation peptide (T^{PE}) consists of 26-112 a.a. residues in length. The PE translocation peptide (T^{PE}) comprises a minimal functional fragment of

(SEQ ID NO: 5)
GWEQLEQCGYPVQRLVALYLAARLSW.

In one embodiment, a PE translocation peptide (T^{PE}) comprises an amino acid sequence that is at least 95%, 97% or 99% identical to SEQ ID NO: 5, 6, 7, 8 or 9. In another embodiment, a T^{PE} comprises an amino acid sequence selected from the group consisting of SEQ ID NO: 5, 6, 7, 8 and 9. In another embodiment, a T^{PE} is PE₂₈₀₋₃₀₅ (SEQ ID NO: 5), PE₂₈₀₋₃₁₃ (SEQ ID NO: NO: 6), PE₂₆₈₋₃₁₃ (SEQ ID NO: NO: 7), PE₂₅₃₋₃₁₃ (SEQ ID NO: 8), or PE₂₅₃₋₃₆₄ (SEQ ID NO: 9; full-length PE domain II).

In one embodiment, the Stx translocation peptide (T^{Stx}) is a functional fragment of Shiga toxin (Stx) subunit A (SEQ ID NO: 10) or Shiga-like toxin I (Sit-I) subunit A (SEQ ID NO: 11). A Stx translocation peptide has translocation function, but no cytotoxic effect of subunit A. Sequence identify between Shiga toxin (Stx) subunit A and Slt-I subunit A is 99% and the two proteins has only one amino acid difference.

In another embodiment, a Stx translocation peptide (T^{Stx}) consists of 8-84 a.a. residues in length. The Stx translocation peptide (T^{Stx}) comprises a minimal functional fragment of

(SEQ ID NO: 12)
LNCHHHAS.

In one embodiment, the Stx translocation peptide (T^{Stx}) comprises an amino acid sequence that is at least 95%, 97% or 99% identical to SEQ ID NO: 12, 13, 14, 15 or 16. In another embodiment, a T^{Stx} comprises an amino acid sequence selected from the group consisting of SEQ ID NOs: 12, 13, 14, 15 and 16. In another embodiment, a T^{Stx} is Stx₂₄₀₋₂₄₇ (SEQ ID NO: 12), Stx₂₄₀₋₂₅₁ (SEQ ID NO: 13), Stx₂₁₁₋₂₄₇ (SEQ ID NO: 14), Stx₂₁₁₋₂₅₁ (SEQ ID NO: 15) or Stx₁₆₈₋₂₅₁ (SEQ ID NO: 16) of Stx subunit A.

A CD40-binding domain permits the fusion protein of the invention to bind to a CD40 receptor on a CD40-expressing cell (e.g., dendritic cell or macrophage). A CD40-binding domain may be one selected from the group consisting of (i) a CD40 ligand (CD40L) or a functional fragment thereof; and (ii) a CD40-specific antibody or a functional fragment thereof. In one embodiment, a functional fragment of CD40L is a truncated CD40L substantially lacking transmembrane and cytoplasmic regions of the full-length CD40L₁₋₂₆₁ protein (SEQ ID NO: 17).

In another embodiment, a CD40L or a functional fragment thereof consists of 154-261 a.a. residues in length. In another embodiment, a CD40L comprises a minimal functional fragment of SEQ ID NO: 19. Further in another embodiment, a CD40L or a functional fragment thereof consists of 154-261 a.a. residues in length and said CD40L comprises a minimal functional fragment of SEQ ID NO: 19.

In one embodiment, a CD40L comprises an amino acid sequence that is at least 95%, 97% or 99% identical to SEQ ID NO: 17, 18 or 19. In another embodiment, the CD40L is selected from the group consisting of CD40L₁₋₂₆₁ (SEQ ID NO: 17), CD40L₄₇₋₂₆₁ (SEQ ID NO: 18) and CD40L₁₀₈₋₂₆₁ (SEQ ID NO: 19).

In another embodiment, a CD40-binding domain is a CD40-specific antibody (or anti-CD40 antibody). A CD40-specific antibody is an antibody specifically recognizing and binding to CD 40 protein. A CD40-specific antibody can bind to CD40 protein on a CD40-expressing cell.

In one embodiment, the CD40-specific antibody comprises a heavy chain variable domain (V_{H}) and a light chain variable domain (V_{L}), wherein the V_{H} comprises the amino acid sequence of SEQ ID NO: 22; and the V_{L} comprises the amino acid sequence of SEQ ID NO: 23.

In another embodiment, the CD40-specific antibody is selected from the group consisting of a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab₂, a minibody and a fully antibody.

In another embodiment, the CD40-binding domain is a CD40-specific scFv (anti-CD40 scFv) comprising a heavy chain variable domain (V_{H}), a light chain variable domain (V_{L}) and a flexible linker (L) connecting the V_{H} and the V_{L}. In one embodiment, the CD40-specific scFv comprises SEQ ID NO: 20 or 21.

In another embodiment, the CD40-binding domain according to the invention is (i) a CD40-specific antibody or a binding fragment thereof, or (ii) a CD40-specific single chain variable fragment (scFv) or a binding fragment thereof; said CD40-specific antibody or said CD40-specific scFv comprising a V_{H} and a V_{L}, wherein: (a) the V comprises SEQ ID NO: 22; and (b) the V_{L} comprises SEQ ID NO: 23.

In another embodiment, the CD40-specific antibody or CD40-specific scFv comprises a V_{H} and a V_{L}, the V_{H} comprising V_{H} CDR1, V_{H} CDR2 and V_{H} CDR3; and the V_{H} comprising V_{L} CDR1, V_{L} CDR2 and V_{L} CDR3, wherein: (i) the V_{H} CDR1, V_{H} CDR2 and V_{H} CDR3 comprises SEQ ID NO: 24, 25 and 26, respectively; and (ii) the V_{L} CDR1, V_{L} CDR2 and V_{L} CDR3 comprises SEQ ID NO: 27, 28 and 29, respectively.

In another embodiment, the CD40-binding domain is a CD40-specific scFv comprising a V_{H} and a V_{L}, wherein: (a) the V_{H} comprises SEQ ID NO: 22; and (b) the V_{L} comprises SEQ ID NO: 23.

In another embodiment, the fusion protein of the invention further comprises an endoplasmic reticulum (ER) retention sequence located at the C-terminal of the antigen, with the proviso that the translocation domain comprises a PE translocation peptide (T^{PE}). The ER retention sequence may comprise SEQ ID NO: 30, 31, 32, 33 or 34. In one embodiment, the ER retention sequence is SEQ ID NO: 30.

In another embodiment, the fusion protein of the invention further comprises a CD28-activating peptide located between the CD40-binding domain and the furin and/or cathepsin L cleavage site.

In one embodiment, the CD28-activating peptide consists of 28-53 a.a. residues in length. In another embodiment, the CD28-activating peptide comprises a minimal functional fragment of SEQ ID NO: 35. In another embodiment, the CD28-activating peptide consists of 28-53 a.a. residues in length and said CD28-activating peptide comprises a minimal functional fragment of SEQ ID NO: 35.

In another embodiment, the CD28-activating peptide comprises an amino acid sequence that is at least 95%, 97% or 99% identical to SEQ ID NO: 35, 36 or 37. In another embodiment, the CD28-activating peptide comprises an amino acid sequence selected from the group consisting of SEQ ID NO: 35, 36 and 37. In another embodiment, the CD28-activating peptide is SEQ ID NO: 35, 36 or 37.

In one embodiment, a pathogen is selected from the group consisting of PRRSV, ASFV, CSFV, PCV2, FMDV, PEDV, SVDV, PRV, TGEV, Mycoplasma hyopneumoniae, NDV, IBV, IBDV, Parvovirus, Poxvirus, Rotavirus, and Influenza Virus. In another embodiment, a pathogen is selected from the group consisting of PRRSV, ASFV, CSFV, PCV2, FMDV and PEDV.

An antigen may comprise one or more antigenic polypeptides derived or selected from PRRSV, ASFV, CSFV, PCV2, FMDV, PEDV, SVDV, PRV, TGEV, Mycoplasma hyopneumoniae, NDV, IBV, IBDV, Parvovirus, Poxvirus, Rotavirus, and Influenza Virus.

In one embodiment, an antigen is a pathogenic antigen selected or derived from the group consisting of ASFV CP204L protein, ASFV E183L protein, CSFV E2 protein, CSFV NS3p protein, PCV2 ORF2 protein, PEDV SI protein, PRRSV ORF6 protein, PRRSV ORF5 protein, PRRSV ORF7 protein, and an antigenic polypeptide thereof. Said antigen may be a fusion antigen comprising at least two antigenic polypeptides. For example, a fusion antigen of ASFV CP204L and E183L, a fusion antigen of CSFV E2 and NS3p, or a fusion of antigens derived from PRRSV and PCV2.

In one embodiment, an antigen comprises an amino acid sequence that is at least 80%, 85%, 90%, 95% or 99% identical to SEQ ID No: 39, 40, 41, 42, 43, 44, 45, 46, 47, 48, 49, 50 or 51. In another embodiment, an antigen is a peptide that is at least 80%, 85%, 90%, 95% or 99% identical to SEQ ID No: 39, 40, 41, 42, 43, 44, 45, 46, 47, 48, 49, 50 or 51. In another embodiment, an antigen is a peptide with an amino acid sequence of SEQ ID No: 39, 40, 41, 42, 43, 44, 45, 46, 47, 48, 49, 50 or 51. Further in another embodiment, an antigen is a peptide of SEQ ID No: 39, 40, 41 42, 43 or 44.

In one embodiment, said antigen comprises at least one epitope for inducing a desired immune response, preferably containing 1 to 50 epitopes, more preferably containing 1 to 20 epitopes.

The antigen may be a single antigen or an antigenic fragment thereof, or a fusion antigen comprising at least two antigenic polypeptides fused together with or without a linker between the two antigenic polypeptides.

A fusion antigen may have a rigid linker, (EAAAAK)ₙ, connecting two different antigenic polypeptides, wherein n is an integer from 0-12, preferably from 2-6, more preferably from 3-4. In other words, the rigid linker comprises 0 to 12 repeats, 2 to 6 repeats or 3 to 4 repeats of the sequence EAAAAK (SEQ ID NO: 38).

The fusion protein of the invention may further comprise a rigid linker between the CD40-binding domain and the furin and/or cathepsin L cleavage site. The rigid linker comprises 0 to 12 repeats of the amino acid sequence EAAAAK (SEQ ID NO: 38). The rigid linker may be (EAAAAK)ₙ, or (SEQ ID NO: 38)ₙ, wherein n is an integer from 0-12, preferably from 2-6, more preferably from 3-4. In one embodiment, the rigid linker comprises 2 to 6 repeats or 3 to 4 repeats of SEQ ID NO: 38.

In another embodiment, the fusion protein of the invention comprises, or consists substantially of, an amino acid sequence that is at least 90%, 95% or 99% identical to SEQ ID NO: 52, 53, 54, 55, 56, 57, 58 or 59. Further in another embodiment, the fusion protein of the invention comprises, or consists substantially of, an amino acid sequence selected from the group consisting of SEQ ID NO: 52, 53, 54, 55, 56, 57, 58 or 59.

The invention further relates to a fusion protein in the manufacture of a medicament for eliciting an antigen-specific cell-mediated immune response and/or an antigen-specific humoral immune response, or for reducing, inhibiting, treating, and/or ameliorating an infectious animal disease caused by a pathogen in an animal in need thereof. In one embodiment, the animal is a non-human animal. In another embodiment, the animal is uninfected by a pathogen.

### Pharmaceutical Composition/Vaccine

The invention further relates to a pharmaceutical composition or a vaccine, which comprises:
(a) the fusion protein of the invention; and
(b) a pharmaceutical acceptable carrier and/or an adjuvant.

The vaccine of the invention is a prophylactic and/or therapeutic vaccine. In one embodiment, the fusion protein is for use in eliciting an antigen-specific cell-mediated immune response and antigen-specific humoral response against a pathogen of interest in an uninfected animal.

The term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18^{th} Ed. Mack Printing Company, 1990, pp. 1289-1329).

Suitable adjuvants include, but not limited to, (1) oil-in-water (o/w) adjuvant (e.g., MONTANIDE™ ISA 15A VG, MONTANIDE® ISA 35 VG, and MONTANIDE™ ISA 28R VG), (2) water-in-oil (w/o) adjuvant (e.g., MONTANIDE™ ISA 61 VG, MONTANIDE™ ISA 71 VG, MONTANIDE™ ISA 71R VG, MONTANIDE™ ISA 761 VG, MONTANIDE™ ISA 78 VG, MONTANIDE™ ISA 763B VG, MONTANIDE™ ISA 660 VG, and Freund's incomplete adjuvant), (3) water-in-oil-in-water (w/o/w) adjuvant (e.g., MONTANIDE™ ISA 201 VG, MONTANIDE™ ISA 206 VG, and MONTANIDE™ ISA 207 VG); (4) aluminum salts adjuvant (e.g., aluminum hydroxide and aluminum phosphate); (5) saponin-based adjuvant (e.g., GPI-0100, Quil A or QS-21); (6) Toll-like receptor (TLR) agonist adjuvant (e.g., Poly I:C, monophosphoryl lipid A and CpG oligonucleotide); and (7) a mixture of above mentioned adjuvants. The CpG oligonucleotide adjuvant includes, but not limited to, class A CpG (i.e., CpG1585, CpG2216 or CpG2336), class B CpG (i.e., CpG1668, CpG1826, CpG2006, CpG2007, CpG BW006 or CpG D-SL01) and class C CpG (i.e., CpG2395, CpG M362 or CpG D-SL03). Another suitable CpG adjuvant is CpG1018. In one embodiment, the adjuvant is a CpG oligonucleotide. In another embodiment, the adjuvant is Freund's incomplete adjuvant (FIA).

The pharmaceutical composition/vaccine may be an enteral or a parenteral dosage form, suitable for transdermal, transmucosal, nasopharyngeal, pulmonary, or direct injection, or for systemic (e.g., parenteral) or local (e.g., intratumor or intralesional injection) administration. Parenteral injection may be via intravenous (i.v), intraperitoneal (i.p.), intramuscular (i.m.), subcutaneous (s.c.) or intradermal (i.d.) routes. The pharmaceutical composition may also be administered orally, e.g., in the form of tablets, coated tablets, dragées, hard and soft gelatine capsules.

The dosage of the fusion protein may vary, depending on the disease to be controlled, the age and the individual condition of the patient and the mode of administration. The dosage may be fitted to individual requirements in each case to obtain a therapeutically effective amount of the fusion protein of the invention to achieve a desired therapeutic response.

For adult patients, a single dosage of about 0.1 to 50 mg, especially about 0.1 to 5 mg, comes into consideration. Depending on severity of the disease and the precise pharmacokinetic profile, the fusion protein may be administered with one dosage unit per week, bi-week, or month, and totally give 1 to 6 dosage units per cycle to satisfy such treatment.

In one embodiment, the invention provides a kit or a packaged pharmaceutical composition comprising a fusion protein of the invention and instructions for using thereof to treat one or more symptoms of an infectious disease caused by a pathogen in an animal in need thereof.

### EXAMPLES

### Methods and Materials

Table 1 shows SEQ ID numbers of and the corresponding polypeptides/fusion proteins.

TABLE 1

| SEQ | | Length |
|---|---|---|
| ID No. | Component name or sequence (N→C) | (aa) |
| 1 | Cleavable linker 1 | 4 |
| | RX¹X²R, wherein X¹ and X² are any amino acid residue. | |
| 2 | Cleavable linker 2 | 6 |
| | RX¹RX²X³R, wherein X¹ and X² are any amino acid | |
| | residue, and X³ is K, F or R. | |
| 3 | Rigid linker 1 (EAAAAK)₃ | 18 |
| 4 | Full length PE | 613 |
| 5 | PE translocation peptide (PE₂₈₀₋₃₀₅, minimal) | 26 |
| 6 | PE translocation peptide (PE₂₈₀₋₃₁₃) | 34 |
| 7 | PE translocation peptide (PE₂₆₈₋₃₁₃) | 46 |
| 8 | PE translocation peptide (PE₂₅₃₋₃₁₃) | 61 |
| 9 | PE translocation peptide (PE₂₅₃₋₃₆₄) | 112 |
| 10 | Full length Shiga toxin (Stx) subunit A | 293 |
| 11 | Full length Shiga-like toxin I (Slt-I) subunit A | 293 |
| 12 | Stx translocation peptide (Stx₂₄₀₋₂₄₇, minimal) | 8 |
| 13 | Stx translocation peptide (Stx₂₄₀₋₂₅₁) | 12 |
| 14 | Stx translocation peptide (Stx₂₁₁₋₂₄₇) | 37 |
| 15 | Stx translocation peptide (Stx₂₁₁₋₂₅₁) | 41 |
| 16 | Stx translocation peptide (Stx₁₆₈₋₂₅₁) | 84 |
| 17 | Full length CD40 ligand (CD40L₁₋₂₆₁) | 261 |
| 18 | Truncated CD40 ligand (CD40L₄₇₋₂₆₁) | 215 |
| 19 | Truncated CD40 ligand (CD40L₁₀₈₋₂₆₁; i.e., 18sCD40L) | 154 |
| 20 | Anti-CD40 scFv (V_{H}-L-V_{L}) | 246 |
| 21 | Anti-CD40 scFv (V_{L}-L-V_{H}) | 246 |
| 22 | V_{H} of the anti-CD40 scFv | 119 |
| 23 | V_{L} of the anti-CD40 scFv | 112 |
| 24 | V_{H} CDRI GFTFSTYGMH | 10 |
| 25 | V_{L} CDR2 GKGLEWLSYISGGSSYIFYADSVRGR | 26 |
| 26 | V_{H} CDR3 CARILRGGSGMDL | 13 |
| 27 | V_{L} CDR1 CTGSSSNIGAGYNVY | 15 |
| 28 | V_{L} CDR2 GNINRPS | 7 |
| 29 | V_{L} CDR3 CAAWDKSISGLV | 12 |
| 30 | ER retention sequence KDEL | 4 |
| 31 | ER retention sequence KKDLRDELKDEL | 12 |
| 32 | ER retention sequence KKDELRDELKDEL | 13 |
| 33 | ER retention sequence KKDELRVELKDEL | 13 |
| 34 | ER retention sequence KDELKDELKDEL | 12 |
| 35 | CD28 consensus sequence | 28 |
| | T¹D²I³Y⁴F⁵C⁶K⁷X⁸E⁹X¹⁰X¹¹Y¹²p¹³p¹⁴p¹⁵Y¹⁶X¹⁷D¹⁸N¹⁹ | |
| | E²⁰K²¹S²²N²³G²⁴T²⁵I²⁶I²⁷H²⁸, wherein | |
| | X⁸ is I or L, X¹⁰ is V, F or A, X¹¹ is M or L, X¹⁷ is L or I. | |
| 36 | CD28-activating peptide (minimal) | 28 |
| 37 | CD28-activating peptide | 53 |
| 38 | Rigid linker EAAAAK | 6 |
| 39 | Antigen ASFV CP204L | 194 |
| 40 | Antigen ASFV E183L | 184 |
| 41 | Antigen ASFV CP204L-E183L | 108 |
| 42 | Antigen CSFV E2 | 331 |
| 43 | Antigen CSFV NS3p | 192 |
| 44 | Antigen CSFV E2-NS3p | 553 |
| 45 | Antigen PCV2 ORF2 | 192 |
| 46 | Antigen PEDV S1 | 708 |
| 47 | Antigen PRRSV D (comprising one repeat of the C- | 60 |
| | terminal portion of PRRSV ORF7) | |
| 48 | Antigen PRRSV DGD (comprising two repeats of the C- | 220 |
| | terminal portion of PRRSV ORF7) | |
| 49 | Antigen PRRSV M (M12) (from fusion of the C-terminal | 165 |
| | portion of NSP 10 and the N-terminal portion of NSP 11) | |
| 50 | Antigen PRRSV R (RSAB) (from fusion of the N- | 62 |
| | terminal portion of PRRSV ORF6 and the N-terminal | |
| | portion of ORF5) | |
| 51 | Antigen PRRSV P (PQAB) (another fusion antigen from | 58 |
| | fusion of the N-terminal portion of PRRSV ORF6 and the | |
| | N-terminal portion of ORF5) | |
| 52 | Fusion protein CD40L-T^{PE}-CP204L-E183L | 652 |
| 53 | Fusion protein CD40L-T^{PE}-CP204L | 438 |
| 54 | Fusion protein CD40L-T^{PE}-E183L | 428 |
| 55 | Fusion protein CD40L-T^{PE}-E2-NS3p | 797 |
| 56 | Fusion protein CP204L-E183L-T^{Stx}-CD40L | 659 |
| 57 | Fusion protein CP204L-T^{Stx}-CD40L | 445 |
| 58 | Fusion protein E183L-T^{Stx}-CD40L | 433 |
| 59 | Fusion protein E2-NS3p-T^{Stx}-CD40L | 804 |

Flow cytometry. Splenocytes were stimulated with an antigenic stimulator (for boosting an immune response against the specific antigen used in the fusion protein of the invention) for 2 hours at 37° C., followed by treating with 50 μg/mL of Brefeldin A and Monensin at 37° C. for 2 hours. The cells were harvested, washed with PBS containing 0.5% BSA, and stained with APC/Cy7-conjugated anti-CD3 antibody, PerCP/Cy5.5-conjugated anti-CD4 antibody, FITC-conjugated anti-CD8 antibody, PE-conjugated anti-CD44 antibody and APC-conjugated anti-CD62L antibody simultaneously. After wash, the cells were permeabilized, fixed and intracellularly stained with PE-conjugated anti-IFN-γ antibody and PE/Cy7-conjugated anti-IL-2 antibody and eFluor450-conjugated anti-TNF-α antibody simultaneously. The intracellular cytokine induction (IFN-γ, IL-2 or TNF-α) of splenocytes with CD8+ or CD4+ memory T cell phenotypes (CD3⁺/CD44^{hi}CD62L^{lo}) were further analyzed by Gallios flow cytometer and Kaluza software.

Enzyme-linked immunospot (ELISpot) assay. Splenocytes were seeded in triplicate in a pretreated murine IFN-γ capturing 96-well plate (CTL IMMUNOSPOT®) at a cell density of 2-10⁵ cells/well in the presence or absence of an antigenic stimulator (for boosting an immune response). The cells were discarded after 24 hours of incubation at 37° C. After wash, the captured IFN-γ was detected by biotin-conjugated anti-murine IFN-γ antibody at room temperature for 2 hours and the IFN-γ-immunospots were developed according to the manufacturer's instructions. The scanning and counting of IFN-γ-immunospots were performed by IMMUNOSPOT® S5 Micro analyzer (CTL). The results were presented as IFN-γ⁺ immunospots per million splenocytes.

Indirect enzyme-linked immunosorbent assay (ELISA). Collected whole blood samples were left undisturbed at 4° C. for 30-60 minutes followed by centrifugation at 5,000 g for 10 minutes to pellet the clot. The serum samples were stored at −20° C. To capture desired antigen-specific antibody, the corresponding antigenic protein or peptide was synthesized and used as a coating protein. The coating protein was diluted in PBS buffer and distributed into 96-well plate at 1 μg/well. After overnight incubation at 4° C., the 96-well plate was blocked with 1% BSA in PBS at 37° C. for 1 hour. The serum samples were thawed, and subsequently 10-fold serial diluted in PBS with 1% BSA. The coated protein was incubated with 100 μl of 1000-fold diluted serum sample at 37° C. for 2 hours. After 4 times washing with phosphate buffered saline TWEEN®-20 (PBST), the antigen-specific antibodies which bound to coating proteins were detected by horseradish peroxidase (HRP)-conjugated goat anti-mouse IgG (at a dilution of 1:10,000, Cat #31430, Thermo Fisher Science) at 37° C. for 30 minutes. Following 4 times of washing with PBST, the HRP-mediated color development was catalyzed in the presence of 100 μL of TMB substrate and quenched by 100 μL of 1 N HCl. The relative titers of antigen-specific antibody in the serum samples were determined by the absorbance at 450 nm.

Statistical analysis. Using t-test, results considered significant when p<0.05.

### Example 1

### Construction of Expression Vectors

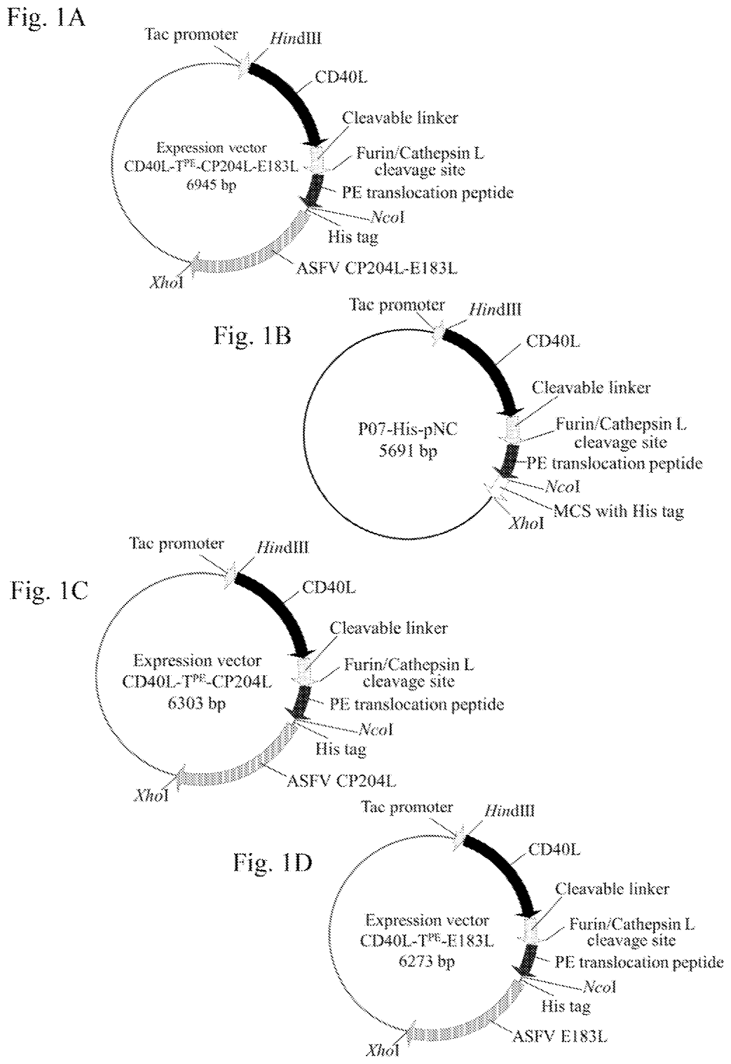

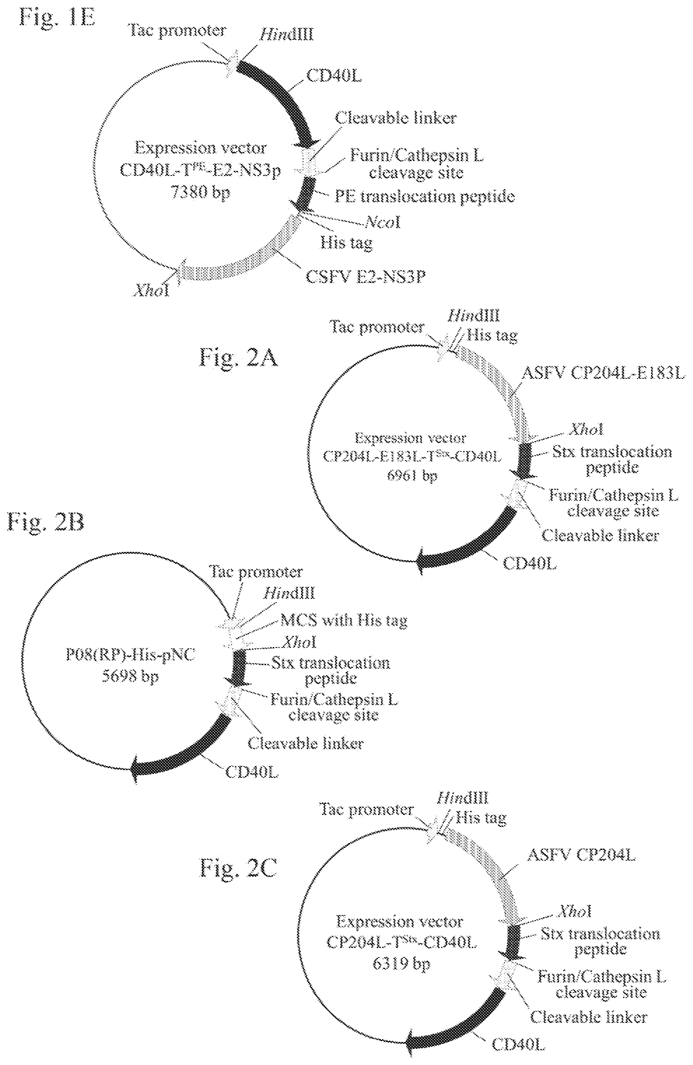

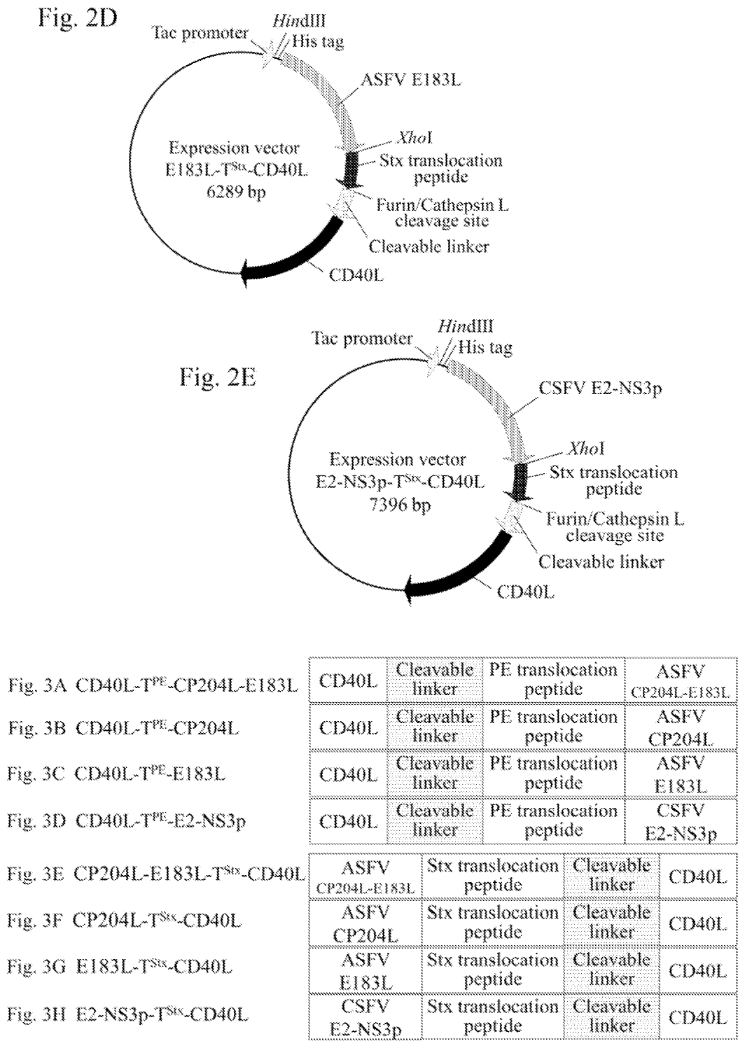

CD40L-T^{PE}-CP204L-E1831, CD40L-T^{PE}-CP204L, CD40L-T^{PE}-E183L and CD40L-T^{PE}-E2-NS3p. The vector CD40L-T^{PE}-CP204L-E183L (FIG. 1A) was constructed to generate CD40L-T^{PE}-CP204L-E183L (SEQ ID NO: 52; FIG. 3A) fusion protein, which comprises: (a) a truncated CD40 ligand CD40L₁₀₈₋₂₆₁ (SEQ ID NO: 19); (b) a cleavable peptide linker, comprising (EAAAAK)₃ (SEQ ID NO: 3) and RX¹RX²X³R (SEQ ID NO: 2; wherein X¹ is A, X² is Y, X³ is K); (c) a PE translocation peptide PE₂₈₀₋₃₀₅ (SEQ ID NO: 5); and (d) a fusion antigen ASFV CP204L-E183L (SEQ ID NO: 41), which comprises the antigen ASFV CP204L (SEQ ID NO: 39) and the antigen ASFV E183L (SEQ ID NO: 40).

Briefly, a DNA fragment encoding ^{HindIII}CD40L-Linker-PE^{NcoI,XhoI,SalI}, comprising the CD40L₁₀₈₋₂₆₁, the cleavable linker and the PE translocation peptide (PE₂₈₀₋₃₀₅), was PCR synthesized, digested by HindIII/SalI and ligated into the plasmid pTAC-MAT-Tag-2 (Catalog No. E5405, Sigma-Aldrich) having HindIII/XhoI cutting sites to obtain the plasmid P07-His-pNC (FIG. 1B). Another DNA fragment encoding said antigen ASFV CP204L-E183L carrying a His tag was inserted into the plasmid P07-His-pNC (FIG. 1B) via NcoI/XhoI sites to generate the expression vector CD40L-T^{PE}-CP204L-E183L (FIG. 1A).

The cleavable linker allows furin and/or cathepsin L protease to cut the fusion protein of the invention for releasing the T^{PE}-CP204L-E183L fragment from the fusion protein.

Using a similar method described above, any other antigen(s) of interest from a pathogen may replace the antigen ASFV CP204L-E183L and be inserted into the plasmid of FIG. 1B to generate an expression vector like FIG. 1A for expressing a fusion protein comprising the antigen(s) of interest.

An expression vector (FIG. 1C) for generating CD40L-T^{PE}-CP204L (SEQ ID NO: 53; FIG. 3B) fusion protein was constructed by replacing the antigen ASFV CP204L-E183L with an antigen ASFV CP204L (SEQ ID NO: 39). Another expression vector (FIG. 1D) for generating CD40L-T^{PE}-E183L (SEQ ID NO: 54; FIG. 3C) fusion protein was constructed by replacing the antigen ASFV CP204L-E183L with an antigen ASFV E183L (SEQ ID NO: 40). Another expression vector (FIG. 1E) for generating CD40L-T^{PE}-E2-NS3p (SEQ ID NO: 55; FIG. 3D) fusion protein was constructed by replacing the antigen ASFV CP204L-E183L with a fusion antigen CSFV E2-NS3p (SEQ ID NO: 44).

Vectors CP204L-E183L-T^{Stx}-CD40L, CP204L-T^{Stx}-CD40L, E183L-T^{Stx}-CD40L, and E2-NS3p-T^{Stx}-CD40L. The vector CP204L-E183L-T^{Stx}-CD40L (FIG. 2A) was constructed to generate CP204L-E183L-T^{Stx}-CD40L (SEQ ID NO: 56; FIG. 3E) fusion protein, which comprises: (a) a fusion antigen ASFV CP204L-E183L (SEQ ID NO: 41), which comprises antigen ASFV CP204L (SEQ ID NO: 39) and antigen ASFV E183L (SEQ ID NO: 40), (b) a Stx translocation peptide Stx₂₁₁₋₂₄₇ (SEQ ID NO: 14), (c) a cleavable peptide linker, comprising RX¹X²R (SEQ ID NO: 1; wherein X¹ is V, X² is A) and (EAAAAK)₃ (SEQ ID NO: 3), and (d) a truncated CD40 ligand CD40L₁₀₈₋₂₆₁ (SEQ ID NO: 19).

Briefly, a DNA fragment encoding ^{HindIII,XhoI}Stx-Linker-CD40L^{SalI}, which comprises the Stx translocation peptide (Stx₂₁₁₋₂₄₇), the cleavable linker and the CD40L₁₀₈₋₂₆₁, was PCR synthesized, digested by HindIII/SalI restriction enzymes, and ligated into the plasmid pTAC-MAT-Tag-2 backbone having HindIII/XhoI cutting sites to obtain the plasmid P08(RP)-His-pNC (FIG. 2B). Another DNA fragment encoding said antigen ASFV CP204L-E183L carrying a His tag was inserted into the plasmid P08(RP)-His-pNC (FIG. 2B) via HindIII/XhoI sites to generate the expression vector CP2041-E183L-T^{Stx}-CD40L (FIG. 2A).

The cleavable linker is vital for the fusion protein of the invention because it allows the fusion protein to be cut by furin and/or cathepsin L protease to release the fragment CP204L-E183L-T^{Stx} from the fusion protein.

Using a similar method described above, any other antigen(s) of interest from a pathogen may replace the antigen ASFV CP204L-E183L and be inserted into the plasmid of FIG. 2B to generate an expression vector like FIG. 2A for expressing a fusion protein comprising the antigen(s) of interest.

An expression vector (FIG. 2C) for generating CP204L-T^{Stx}-CD40L (SEQ ID NO: 57; FIG. 3F) fusion protein was constructed by replacing the antigen ASFV CP204L-E183L with antigen ASFV CP204L (SEQ ID NO: 39). An expression vector (FIG. 2D) for generating E183L-T^{Stx}-CD40L (SEQ ID NO: 58; FIG. 3G) fusion protein was constructed similarly by replacing the antigen ASFV CP204L-E183L with antigen ASFV E183L (SEQ ID NO: 40). Another expression vector (FIG. 2E) for generating E2-NS3p-T^{Stx}-CD40L (SEQ ID NO: 59; FIG. 3H) fusion protein was constructed by replacing the antigen ASFV CP204L-E183L with antigen CSFV E2-NS3p (SEQ ID NO: 44).

### Example 2

### Protein Expression

E. coli BL21 cells harboring the protein expression vector CD40L-T^{PE}-E2-NS3p were grown in ZY media (10 g/L tryptone and 5 g/L yeast extract) containing a selection antibiotic at 37° C. When the culture reached an early log phase (OD₆₀₀=2 to 5), the expression of the fusion protein was induced by isopropyl-1-thio-β-D-galactopyranoside (IPTG) (0.5 to 2 mM). Cells were harvested after 4 hours of the IPTG induction and disrupted by sonication. The inclusion bodies were isolated and solubilized in solubilization buffer (6 M guanidine hydrochloride, 20 mM potassium phosphate, 500 mM NaCl, 20 mM imidazole, 1 mM DTT, pH 7.4) to recover overexpressed fusion proteins. After purification, the refolding of the fusion protein was performed by dialysis against 20- to 50-fold volume of dialysis buffer (10 mM PBS) at 4° C. overnight. The refolded fusion proteins were subject to SDS-PAGE analyses under reduced (with dithiothreitol; +DTT) and non-reduced (without dithiothreitol; −DTT) conditions to evaluate whether they were properly refolded.

The fusion proteins CD40L-T^{PE}-CP204L-E183L, CD40L-T^{PE}-CP204L, CD40L-T^{PF}-E183L, CP204L-E183L-T^{Stx}-CD40L, CP204L-T^{Stx}-CD40L, E183L-T^{Stx}-CD40L, and E2-NS3p-T^{Stx}-CD40L are expressed, purified, and refolded by using the same method described above.

### Example 3

### Immunogenicity Analyses of Fusion Proteins

CD40L-T^{PE}-E2-NS3p fusion protein was chosen as a representative of the fusion proteins of the invention and subjected to immunogenicity analyses to evaluate the biological activities of the fusion proteins. Table 2 shows the animal groups, the doses of the fusion protein for vaccination, and the dosing schedule. “V”: vaccinated; “-”: not vaccinated.

TABLE 2

| | Fusion protein | Day | Day | Day | Day |
|---|---|---|---|---|---|
| Group | (μg/dose) | 0 | 7 | 14 | 21 |
| A | 0 | — | — | — | Sacrifice |
| B | 50 | V | V | V | |
| C | 100 | V | V | V | |
| D | 100 | V | V | — | |
| E | 100 | V | — | V | |

Preparation of vaccine: A fusion protein of the invention, dissolved in phosphate-buffered saline, was mixed with an equal volume of the Freund's incomplete adjuvant (FIA) to form an emulsion. The final formulation contained 0.5 mg of the fusion protein/mL and 50% (v/v) of Freund's incomplete adjuvant.

C57BL/6NCrlBltw female mice (5-6 weeks old) were randomly divided into six groups (n=5 per group): group A (placebo), group B (50 μg of the test fusion protein), and group C to group E (100 μg of the test fusion protein). The placebo group received PBS s.c. on Days 0, 7 and 14. Mice in the vaccination groups (B to E) were injected s.c. with the fusion protein adjuvanted with the Freund's incomplete adjuvant according to the dosing schedule in Table 2. Serums were collected on Days 0, 7, 14 and 21 to determine the antigen-specific humoral immune response. On Day 21, the animals were sacrificed. The splenocytes were harvested and cultured to determine the antigen-specific cell-mediated immune response.

The splenocytes were used to analyze intracellular cytokine induction (IFN-γ, IL-2 and TNF-α) in memory T cells by using flow cytometry. Additionally, the frequency of IFN-γ-secreting splenocytes was analyzed by using Enzyme-linked immunospot (ELISpot) assay. The levels of serum antigen-specific antibody in the blood samples were analyzed by using ELISA.

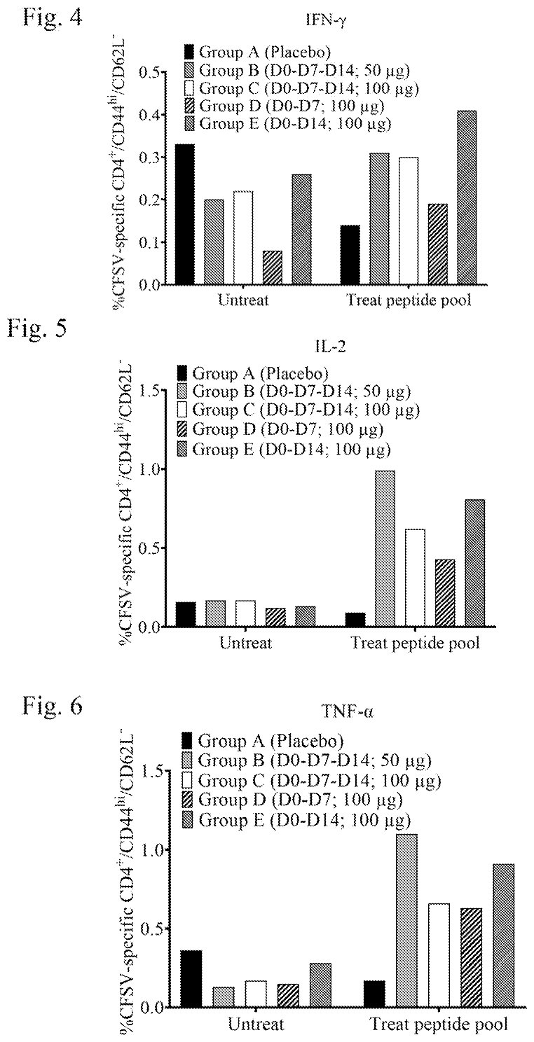

FIGS. 4-6 show the results of IFN-γ, IL-2 and TNF-α inductions, respectively, after the splenocytes being stimulated with or without an antigenic stimulator (a short peptide pool covering the sequence of the antigen CSFV E2). On Day 21, the levels of IFN-γ, IL-2 and TNF-α induction in the CD4⁺ memory T cells from the groups receiving different doses or different regimens of CD40L-T^{PE}-E2-NS3p were observed and compared to the placebo group.

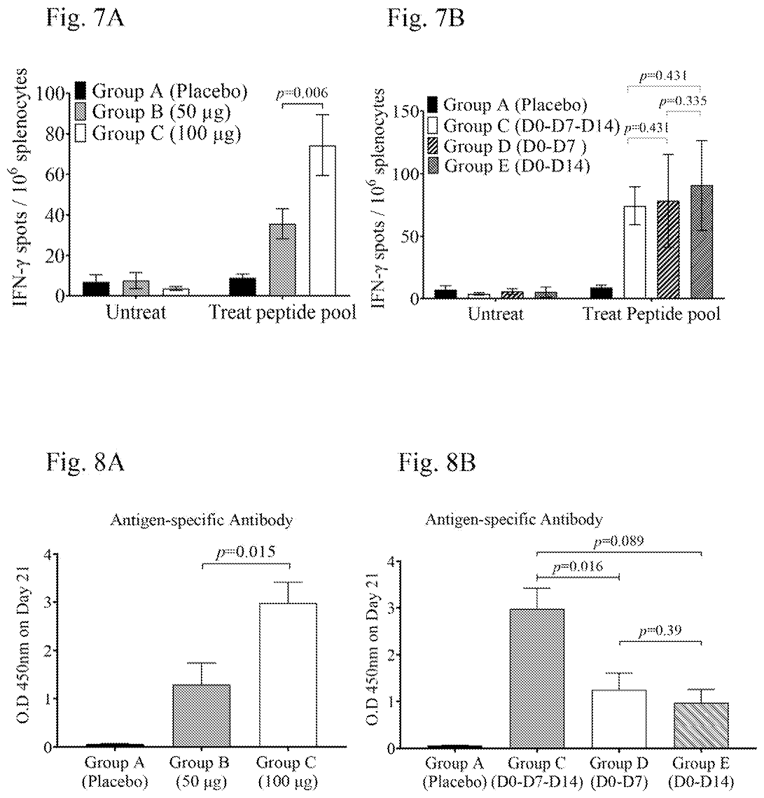

FIGS. 7A-B show the results of IFN-γ⁺ immunospots after the splenocytes, obtained from the animals on Day 21, being stimulated with or without an antigenic stimulator as aforementioned in vitro. FIG. 7A shows a significant increase in the population of IFN-γ-secreting splenocytes obtained from the animals of B and C groups on Day 21, in which the groups B and C animals were vaccinated with 50 μg and 100 μg of CD40L-T^{PE}-E2-NS3p, respectively, on Days 0, 7 and 14. Noticeably, the high-dose group exhibited a more potent activity in inducing IFN-γ-secreting splenocytes as compared to the low-dose group (p==0.006). FIG. 7B shows a significant increase in the population of IFN-γ-secreting splenocytes in the animal groups of C, D and E as compared to the placebo group. The animals in the groups of C, D and E were all vaccinated with 100 μg of CD40L-T^{PE}-E2-NS3p fusion protein, but on different schedules (D0-D7-D14, D0-D7 and D0-D14, respectively).

The fusion protein of the invention can effectively elicit an antigen-specific antibody response. FIGS. 8A-B show the serum antibody levels on Day 21 as measured by ELISA. CD40L-T^{PE}-E2-NS3p fusion protein was used to coat the ELISA plates as a capture antigen.

FIG. 8A shows serum antigen-specific antibody levels in the animal groups A, B and C on Day 21. The animals in the groups B and C were vaccinated with 50 μg and 100 μg of CD40L-T^{PE}-E2-NS3p, respectively, on Days 0, 7 and 14. The serum levels of the antigen-specific antibody in the vaccinated groups B and C were both higher than that of the placebo group, and the high-dose group elicited a stronger antibody response than the low-dose group (p=0.015).

FIG. 8B shows serum antigen-specific antibody levels in the animal groups A, C, D, and E on Day 21. The animals in the groups C, D, and E were all vaccinated with 100 μg of CD40L-T^{PE}-E2-NS3p but on different regimens (D0-D7-D14, D0-D7 and D0-D14, respectively). The group vaccinated by three doses and the groups vaccinated by two doses both showed a significant increase in the serum antigen-specific antibody titers. Noticeably, the third dose could elicit a significantly higher antigen-specific antibody response than the two doses.

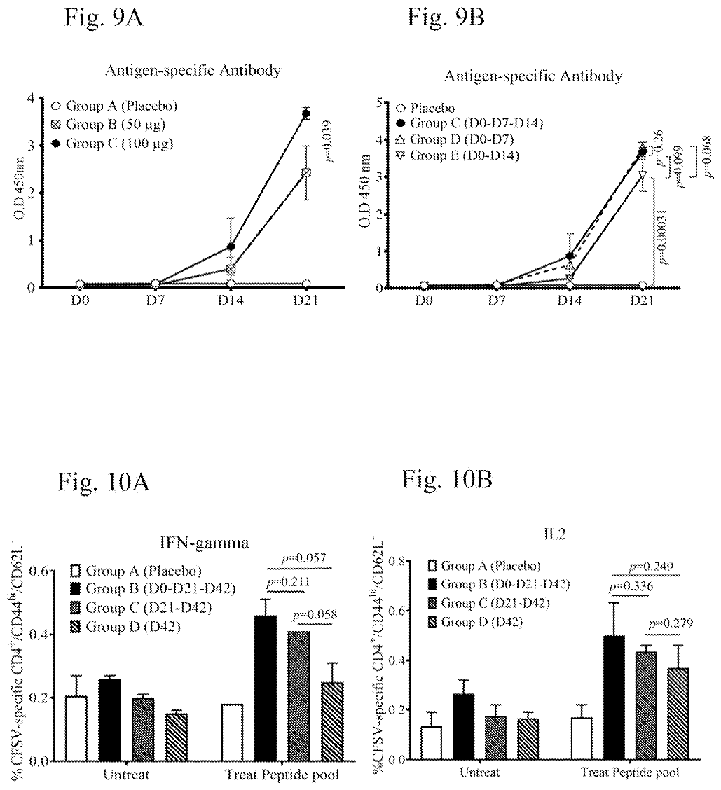

FIG. 9A-B show the serum antibody levels on Day 21 in the animal groups. Similar to FIGS. 8A-B, the antigen-specific antibody levels were measured by ELISA except the coating protein being E2-NS3p peptide (capture antigen), rather than the CD40L-T^{PE}-E2-NS3p fusion protein. The results were consistent with the finding in FIGS. 8A-B. The serum levels of the antigen-specific antibody in groups B and C on Day 21 were higher than that in the placebo group, and the high-dose group elicited a stronger antibody titer than the low-dose group.

The fusion proteins CD40L-T^{PE}-CP204L-E183L, CD40L-T^{PE}-CP204L, CD40L-T^{PE}-E183L, CP204L-E183L-T^{Stx}-CD40L, CP204L-T^{Stx}-CD40L, E183L-T^{Stx}-CD40L, and E2-NS3p-T^{Stx}-CD40L are to be subjected to immunogenicity analyses. Based on the same mechanism of action, the antigen-carrying fusion proteins of the invention are expected to induce potent immune responses against a target antigen, leading to induction of antigen-specific antibody, T cell activation, and induction of IFN-γ, IL-2 and TNF-α.

### Example 4

### Immunogenicity Analyses of Fusion Proteins with an Extended Dosing Schedule

The effect of CD40L-T^{PE}-E2-NS3p fusion protein in inducing an immune response was also evaluated under an extended dosing schedule. Table 3 shows animal groups and the dosing schedule of each group. “V”: vaccinated; “-”: not vaccinated.

TABLE 3

| | Fusion protein | Day | Day | Day | Day |
|---|---|---|---|---|---|
| Group | (μg/dose) | 0 | 21 | 42 | 63 |
| A | 0 | — | — | — | Sacrifice |
| B | 100 | V | V | V | |
| C | 100 | — | V | V | |
| D | 100 | — | — | V | |

Except the extended dosing schedule and the mouse groups, the preparation of the vaccines, the mice, administration route, and analytical methods were like those described in Example 3.

Briefly, C57BL/6NCrlBltw female mice (5-6 weeks old) were randomly divided into four groups (n=5 per group): group A (placebo), groups B, C and D (100 μg fusion protein). The placebo group received PBS s.c. on Days 0, 21 and 42. Mice in the groups B, C, and D were vaccinated s.c with the test fusion protein adjuvanted with the FIA according to the respective dosing schedule of the groups B to D in Table 3. Serums were collected weekly to determine antigen-specific humoral immune response. On Day 63, the animals were sacrificed, and splenocytes were harvested and cultured to determine antigen-specific cell-mediated immune response by using the analytical methods described in Example 3.

FIGS. 10A-C show the results of IFN-γ, IL-2 and TNF-α inductions after the splenocytes, obtained from the animals on Day 63, being stimulated with or without an antigenic stimulator (a short peptide pool covering the sequence of the antigen CSFV E2). The levels of IFN-γ, IL-2 and TNF-α inductions in the CD4⁺ memory T cells from the groups receiving different regimens of CD40L-T^{PE}-E2-NS3p were observed and compared to the placebo group.

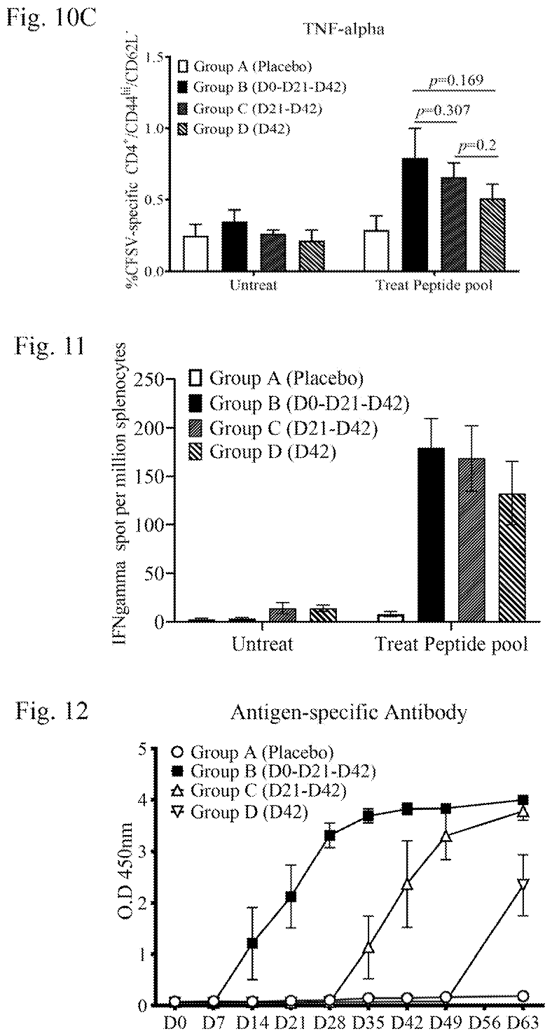

FIG. 11 shows the results of IFN-γ⁺ immunospots after the splenocytes, obtained from the animals on Day 63, being treated with or without an antigenic stimulator as aforementioned. A significant increase in the population of IFN-γ-secreting splenocytes was observed in all the animal groups receiving different regimens of CD40L-T^{PE}-E2-NS3p as compared to the placebo group. One shot of the fusion protein of the invention was sufficient to induce an increase in the population of IFN-γ-secreting splenocytes (FIG. 11, Group D).

FIG. 12 shows the levels of serum antigen-specific antibody in the animal groups A, B, C and D, respectively. The animals in the groups B, C and D were vaccinated with CD40L-T^{PE}-E2-NS3p on different regimens (D0-D21-D42, D21-D42 and D42, respectively). The coating protein (capture antigen) used in the ELISA was the antigenic peptide E2-NS3p. The results were consistent with the finding above. One dose of the fusion protein CD40L-T^{PE}-E2-NS3p was enough to induce an antigen-specific antibody response. Noticeably, in the three-doses-group a prolonged period of antibody response lasting for at least 21 days was observed after the third dose on Day 42.

In summary, the fusion proteins of the invention could elicit a potent T cell immune response, increase the expression of IFN-γ, IL-2 and TNF-α, and generate an antigen-specific antibody response.

All references cited and discussed in this specification are incorporated herein by reference in their entireties and to the same extent as if each reference was individually incorporated by reference.

1. A fusion protein comprising:
(a) a CD40-binding domain, which is a CD40 ligand (CD40L) or a functional fragment thereof comprising an amino acid sequence that is at least 95% identical to SEO ID NO: 19, said CD40L or functional fragment thereof consisting of 154-261 amino acid residues in length;
(b) an antigen of a pathogen;
(c) a translocation domain, located between the CD40-binding domain and the antigen, said translocation domain being selected from the group consisting of:
(c1) a Shiga toxin (Stx) translocation peptide; and
(c2) a Pseudomonas Exotoxin A (PE) translocation peptide; and
(d) a furin and/or cathepsin L cleavage site, located between the CD40-binding domain and the translocation domain,
wherein the pathogen is at least one selected from the group consisting of Classical Swine Fever Virus (CSFV), African Swine Fever Virus (ASFV), Porcine Circovirus 2 (PCV2), Porcine Reproductive and Respiratory Syndrome Virus (PRRSV), Porcine Epidemic Diarrhea Virus (PEDV), Foot-and-Mouth Disease Virus (FMDV), Swine Vesicular Disease Virus (SVDV), Pseudorabies Virus (PRV), Transmissible Gastroenteritis Virus (TGEV), Mycoplasma hyopneumoniae, Newcastle Disease Virus (NDV), Infectious Bronchitis Virus (IBV), Infectious Bursal Disease Virus (IBDV), Parvovirus, Poxvirus, Rotavirus, and Influenza Virus; and further wherein when the translocation domain is the Stx translocation peptide, the antigen is located at the N-terminal of the fusion protein; and when the translocation domain is the PE translocation peptide, the CD40-binding domain is located at the N-terminal of the fusion protein.

2. The fusion protein of claim 1, wherein the translocation domain is the PE translocation peptide, the PE translocation peptide consisting of 26-112 amino acid residues in length and comprising an amino acid sequence of SEO ID NO: 5.

3. The fusion protein of claim 1, wherein the translocation domain is the PE translocation peptide consisting of 26-112 amino acid residues in length and comprising an amino acid sequence that is at least 95% identical to SEQ ID NO: 5, 6, 7, 8 or 9.

4. The fusion protein of claim 1, wherein the translocation domain is the Stx translocation peptide, the Stx translocation peptide consisting of 8-84 amino acid residues in length and comprising an amino acid sequence of SEQ ID NOs:12.

5. The fusion protein of claim 1, wherein the translocation domain is the Stx translocation peptide consisting of 8-84 amino acid residues in length and comprises an amino acid sequence that is at least 95% identical to SEQ ID NOs: 12, 13, 14, 15 or 16.

6. The fusion protein of claim 1, wherein the furin and/or cathepsin L cleavage site comprises an amino acid sequence of SEQ ID NO: 1 or 2.

7. The fusion protein of claim 1, further comprising a peptide linker, said peptide linker comprising the furin and/or cathepsin L cleavage site located between the CD40-binding domain and the translocation domain.

8. The fusion protein of claim 1, wherein the CD40L or the functional fragment thereof comprises an amino acid sequence selected from the group consisting of SEQ ID NO. 17, 18 and 19.

9. The fusion protein of claim 1, wherein the CD40L consists of 154-261 amino acid residues in length and comprises an amino acid sequence that is at least 97% identical to SEQ ID NO: 17, 18 or 19.

10. (canceled)

11. (canceled)

12. The fusion protein of claim 2, further comprising an endoplasmic reticulum (ER) retention sequence located at the C-terminus of the antigen.

13. The fusion protein of claim 3, further comprising a CD28-activating peptide located between the CD40-binding domain and the furin and/or cathepsin L cleavage site, wherein the CD28-activating peptide has a length of 28-53 amino acid residues and comprises an amino acid sequence selected from the group consisting of SEQ ID NO: 35, 36 and 37.

14. A pharmaceutical composition comprising:
(a) the fusion protein of claim 1; and
(b) a pharmaceutical acceptable carrier and/or an adjuvant.

15. A method for eliciting an antigen-specific, cell-mediated immune response, or for reducing, inhibiting, treating, and/or ameliorating an infectious animal disease caused by a pathogen in an animal in need thereof, comprising:
administering a therapeutically effective amount of the fusion protein of claim 1 to the animal in need thereof, and thereby eliciting the antigen-specific, cell-mediated immune response, or reducing, inhibiting, treating, and/or ameliorating the infectious animal disease caused by the pathogen in the animal in need thereof.

16. The fusion protein of claim 3, wherein the PE translocation peptide comprises the amino acid sequence selected from the group consisting of SEQ ID NOs: 5, 6, 7, 8 and 9.

17. The fusion protein of claim 5, wherein the Stx translocation peptide comprises an amino acid sequence selected from the group consisting of SEQ ID NOs: 12, 13, 14, 15 and 16.

18. A fusion protein comprising:
(a) a CD40-binding domain, which is a CD40 ligand (CD40L) or a functional fragment thereof comprising an amino acid sequence that is at least 95% identical to SEQ ID NO: 19, said CD40L or functional fragment thereof consisting of 154-261 amino acid residues in length;
(b) an antigen of a pathogen;
(c) a translocation domain, located between the CD40-binding domain and the antigen, said translocation domain being selected from the group consisting of:
(c1) a Shiga toxin (Stx) translocation peptide, comprising an amino acid sequence of SEQ ID NO: 12; and
(c2) a Pseudomonas Exotoxin A (PE) translocation peptide, comprising an amino acid sequence of SEQ ID NO: 5; and
(d) a furin and/or cathepsin L cleavage site, located between the CD40-binding domain and the translocation domain,
wherein the pathogen is at least one selected from the group consisting of Classical Swine Fever Virus (CSFV), African Swine Fever Virus (ASFV), Porcine Circovirus 2 (PCV2), Porcine Reproductive and Respiratory Syndrome Virus (PRRSV), Porcine Epidemic Diarrhea Virus (PEDV), Foot-and-Mouth Disease Virus (FMDV), Swine Vesicular Disease Virus (SVDV), Pseudorabies Virus (PRV), Transmissible Gastroenteritis Virus (TGEV), Mycoplasma hyopneumoniae, Newcastle Disease Virus (NDV), Infectious Bronchitis Virus (IBV), Infectious Bursal Disease Virus (IBDV), Parvovirus, Poxvirus, Rotavirus, and Influenza Virus; and further wherein when the translocation domain is the Stx translocation peptide, the antigen is located at the N-terminal of the fusion protein; and when the translocation domain is the PE translocation peptide, the CD40-binding domain is located at the N-terminal of the fusion protein.

19. The fusion protein of claim 18, wherein the CD40L or the functional fragment thereof comprises an amino acid sequence selected from the group consisting of SEQ ID NO. 17, 18 and 19.

20. A method for eliciting an antigen-specific, cell-mediated immune response, or for reducing, inhibiting, treating, and/or ameliorating an infectious animal disease caused by a pathogen in an animal in need thereof, comprising:
administering a therapeutically effective amount of the fusion protein of claim 18 to the animal in need thereof, and thereby eliciting the antigen-specific, cell-mediated immune response, or reducing, inhibiting, treating, and/or ameliorating the infectious animal disease caused by the pathogen in the animal in need thereof.

21. The fusion protein of claim 1, wherein the antigen is a fusion antigen comprising at least two antigenic polypeptides.

22. The fusion protein of claim 1, wherein the antigen is selected from the group consisting of ASFV CP204L protein, ASFV E183L protein, a fusion antigen of ASFV CP204L and E183L, and a fusion antigen of CSFV E2 and NS3p.
